(19) United States
(12) Patent Application Publication
(10) Pub. No.: US 2019/0100768 A1
(43) Pub. Date: Apr. 4, 2019

(54) Tospovirus Resistant Plants and Methods Thereof

(71) Applicants: Tierra Seed Science Pvt. Ltd., Hyderabad, Telangana (IN); SEQ ID 1.0 INC, Toronto (CA)

(72) Inventors: Parthasarathi Bhattacharya, Hyderabad, Telangana (IN); Suresh Atluri, Hyderabad, Telangana (IN); Surinder Kumar Tikoo, Hyderabad, Telangana (IN); Charles Pick, Toronto (CA)

(21) Appl. No.: 15/555,937

(22) PCT Filed: Aug. 28, 2017
(86) PCT No.: PCT/IB2017/055151

§ 371 Date: Sep. 5, 2017

(51) Int. Cl. C12N 15/82; A01H 6/82; A01H 5/08

(52) U.S. Cl. CPC C12N 15/8283; A01H 5/08; A01H 6/825

(57) ABSTRACT

The present invention provides tomato plants exhibiting resistance to TOSPO (GBNV) Virus exhibiting the traits of healthy and evenly ripened fruits without brown spots, reduced wilting of the top portion of the tomato plants and reduced dark spots on the leaves and stems of the tomato plant, and materials useful for improving tomato yield. In particular, the present invention provides a path to development of hybrid tomato plants exhibiting resistance to TOSPO virus infection, seed of hybrid tomato plants exhibiting TOSPO virus resistance, and methods of producing the same. Methods described herein involve the use of marker assisted selection and marker assisted backcrossing utilizing molecular markers associated with a tomato TOSPO Resistance phenotype.

### FIELD OF INVENTION

The present invention relates to the field of agricultural biotechnology more specifically to the development of disease resistant tomato lines.

### BACKGROUND

Viruses of the genus TOSPO cause significant worldwide crop losses. Tospoviruses have a tripartite RNA genome of ambisense polarity. The Tospovirus genus includes plant viruses that infect a wide range of plant species including vegetable, fruit and ornamental crops. The viruses belonging to this genus are popularly described as tospovirus and tomato spotted wilt virus is a type of species belonging to this genus. Tospoviruses are distributed in tropical, subtropical and temperate regions throughout the Northern Hemisphere, Western Europe and Asia. Ground nut bud necrosis virus (GBNV) is currently recognized as the most economically important tospovirus in South Asia & South East Asia. Losses due to GBNV alone have been estimated at more than US$89 million per annum in Asia.

The transmission of the disease is by the vector Thrips palmi. The disease is affecting tomato crop cultivation to the tune of 350,000 hectares per year in India, especially in Kharif season. Control of Tospoviruses remains problematic. Cultural practices and varietal selection have proven effective in minimizing losses due to tomato spotted wilt virus (TSWV) in some field crops. A series of risk factors including prior history, planting date, cultivar selection and plant and row spacing have been identified as critical factors in peanuts. In other high-risk areas, such as Hawaii, highly susceptible crops cannot be grown profitably. In greenhouse grown crops, such as floral crops, extreme measures including screening of production areas with fine-meshed cloth, preventative thrips management strategies and use of propagation material shown to be free of TSWV and impatiens necrosis spot virus (INSV) are necessary for control of these viruses. While forms of resistance have been introduced into various crops, they have nearly always been overcome by the rapid occurrence of resistance-breaking strains of the virus. TSWV is thought to exist in nature as a complex heterogeneous mixture of distinct isolates that can exchange genetic information through re-assortment of genome segments.

This provides a readily available reservoir of genetic information to facilitate adaptation. For the reasons stated above control of the disease is a challenge so far. Various resistant genes were identified and designated as Swa1, Swb1, Sw2, Sw3, and Sw4, Sw-5, Sw6, Sw7 from wild source of tomato (S. peruvianum) against TOSPO virus. However, these resistance genes are not effective to control the GBNV disease in India. Chemical control measures have not been able to contain the disease and farmers are suffering yield loss on account of the disease outbreak. The estimated loss (Kunkalikar, S. R. et al (2011) is around 80% per season of the crop. The calculated loss to the farmer in India is approximate of US$25 million if approximately 50% of acreage gets affected by the disease i.e. 175,000 hectares. As known genetic resistance and chemical measures are not effective in control of the disease, there is a need to develop an edible and cultivable tomato line resistant to GNBV.

### OBJECT OF THE INVENTION

The object of the invention is for an edible and cultivable tomato line resistant to GNBV, and related method for developing such line.

The invention also relates to a cultivable & edible F1 hybrid variety resistant to GBNV, and related method for developing such hybrid line.

The invention relates to the identification of DNA markers linked to GBNV resistance trait and use of marker assisted breeding for selection of resistant edible and cultivable tomato line or F1 hybrid variety

### BRIEF DESCRIPTION OF DRAWINGS AND FIGURES

FIG. 1 shows the flow chart of breeding interspecific cross up to BC2F2.

FIG. 2 shows the flow chart of breeding BC2F3 to current status.

FIG. 3 depicting the frequency distribution of TOSPO (GBNV) disease over generations.

FIG. 4 photomicrographs of the disease scoring chart.

FIG. 5 depicts the distribution of BC2F3 lines with different recipient allele frequency based on 2455 SNP.

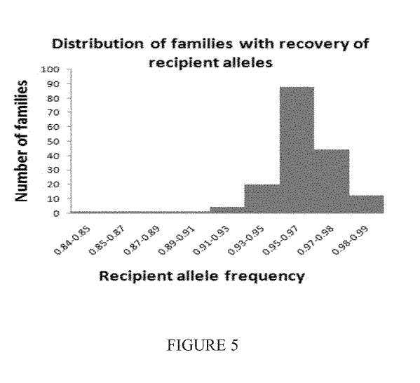

FIG. 6 showing BC2F6 & BC2F7 families (RP×SP1)×SP2 indicating trait linked markers in resistant progenies.

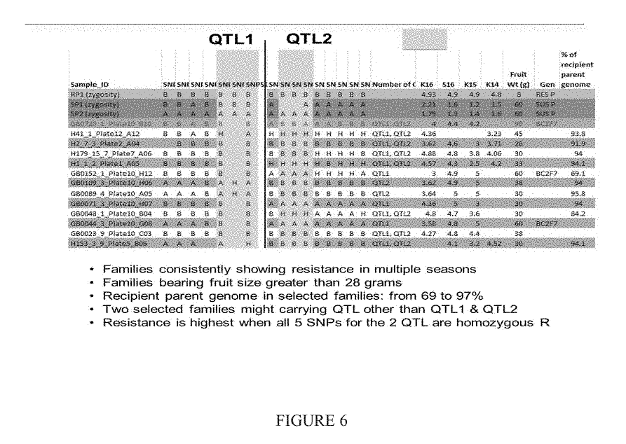

### DETAILED DESCRIPTION OF THE INVENTION

Accordingly, the present invention provides an edible and cultivable tomato line resistant to GBNV, and related method for developing such line.

The invention also relates to development of cultivable & edible F1 hybrid variety resistant to GBNV, and related method thereof.

The invention relates to the identification of markers and use of marker assisted breeding for selection of GBNV resistant edible and cultivable tomato line.

The donor is selected from Solanum peruvianum line obtained from World Vegetable Centre—Asian Vegetable Research and Development Center (AVRDC), Taiwan.

Susceptible cultivated lines of tomato used in this work are collections made by Tierra Seed Science from known Indian sources following proper legal process Cultivated lines used were SP-1 (Susceptible parent 1-Sel 22, purchased from Indian Institute of Horticultural Research Bengaluru (IIHR), a commercial variety since 1985). SP-2 (Susceptible line)—collected from AVRDC carrying Ty 2 gene that gives partial resistance against TyLCV (Tomato Yellow Leaf Curl Virus) but susceptible to TOSPO (GBNV).

The invention provides a method for producing a tomato plant, wherein the tomato plant exhibits TOSPO Virus resistance, comprising:

(a) providing a donor tomato plant, wherein the donor plant exhibits TOSPO virus resistance;

(b) transferring at least one nucleic acid from the first tomato plant to one or more tomato plants, wherein the recipient tomato plants do not exhibit TOSPO virus resistance, and wherein the transfer results in the introduction of genomic material from the donor tomato plant in a corresponding region of one or more recipient tomato plants; and

(c) identifying and selecting, from the one or more recipient tomato plants, a tomato plant that comprises within its genome at least one quantitative trait locus (QTL) for TOSPO virus resistance, wherein the QTL is selected from the group consisting of: QTL1, QTL2 and TOSPO viral resistance related traits thereof, and wherein QTL1 is indicated by a genomic region on chromosome 1, QTL2 is indicated by a genomic region on chromosome 2

The method for producing the disease resistant tomato plant further comprising the step of selecting a recipient tomato plant which is originally susceptible to TOSPO Viruses Some of the post infection common characteristics traits of the recipient tomato plant which is susceptible to TOSPO are deformed shape and uneven ripening of the fruit, wilting of the top portion of the plant, dark brown spots on the leaves and dark brown patches on stems of the plants.

The transfer of one or more nucleic acids is performed by crossing the donor tomato plant with a recipient tomato plant to produce progeny tomato plants comprising at least one QTL for TOSPO virus resistance as in introgression, and wherein identification and selection is performed on one or more progeny plants.

The method of transfer of one or more nucleic acids is based on selecting the TOSPO (GBNV) resistant plants from progenies of an interspecific cross. The crossing was was made possible using embryo rescue technique to overcome incompatibility.

Further, the method of the invention is performed by detecting the marker in DNA isolated from the recipient tomato plants or from one or more of progeny plants.

The method of the invention comprises selecting a recipient tomato plant that comprises within its genome at least two QTLs selected from the group consisting of QTL1, QTL 2 and virus-resistance related parts thereof.

The method of the invention comprises selecting a recipient tomato plant that comprises QTL1 and QTL2 within its genome.

The method of the invention comprises selecting a recipient tomato plant that comprises within its genome a QTL or a viral resistance related part thereof, and at least one QTL selected from the group consisting of QTL1, QTL2 and related parts thereof.

The method of the invention further comprises a step of crossing a recipient tomato plant with a plant of a cultivated variety of tomato to produce progeny plants.

The method of the invention comprises the recipient tomato plant as same as that of the plant of the cultivated variety of tomato to which the recipient tomato plant is crossed.

The resistance provided against by the method of the invention is for a Groundnut Bud Necrosis Virus (GBNV) which is a TOSPO virus.

The invention is for a tomato plant exhibiting TOSPO virus resistance, and the said tomato plant or part thereof comprises a QTL selected from the group consisting of QTL1, QTL2 and TOSPO virus related parts thereof, wherein the QTLs or viral resistance-related parts thereof are not present in the natural genetic background of the edible tomato plants.

The tomato plant exhibiting TOSPO virus resistance, is produced by back crossing the tomato plant BC 1 (back cross 1) with a tomato plant that exhibits commercially desirable characteristics, wherein the resulting hybrid plant comprises at least one QTL selected from the group consisting of QTL1, QTL2 and TOSPO virus resistance related parts thereof, wherein at least one QTL is not present in the natural background of either the tomato plant selected in step 1 (c) or the tomato plant exhibiting commercially desirable characteristics.

Standard embryo rescue method was employed to overcome sexual incompatibility between S. peruvianum & S. lycopersicum, and to raise the seven day old fertilized ovules on tissue culture medium.

Scoring of the disease symptoms (phenotyping) was performed in a hot spot location for TOSPO (GBNV) disease and were categorized into 5 types (1) whole plant diseased (score 1); (2) diseased with few green stem and leaves (score 2); (3) 50% plant covered-may have 1 or 2 fruit (score 3); (4) Except for the top leaves, stem and other plant parts are healthy (score 4); (5) Healthy plants (score 5) using the phenotypic symptoms observed in the field at young and adult plant stages, e.g 40, 60, 80 days after transplantation in the field.

### EXAMPLES

The following examples are for the purpose of illustration of the invention and are not intended in any way to limit the scope of the invention.

Materials:

Wild type Solanum peruvianum line was obtained from World Vegetable Centre—Asian Vegetable Research and Development Center (AVRDC), Taiwan. Susceptible cultivated lines of tomato used in this work are collected from known germplasm resources. The cultivated lines used were SP-1 (Susceptible parent 1-Sel 22, purchased from Indian Institute of Horticultural Research Bengaluru (IIHR), a commercial line since 1985). SP-2 (Susceptible line)—A Ty 2 line collected from AVRDC but susceptible to TOSPO.

### Example 1

Development and Determination of Disease Resistant Phenotype:

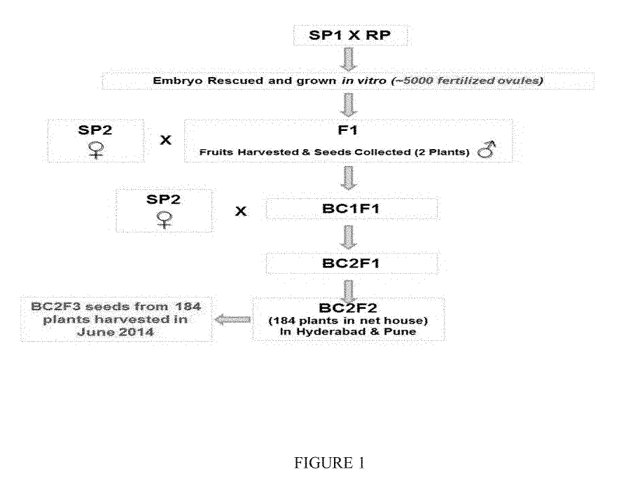

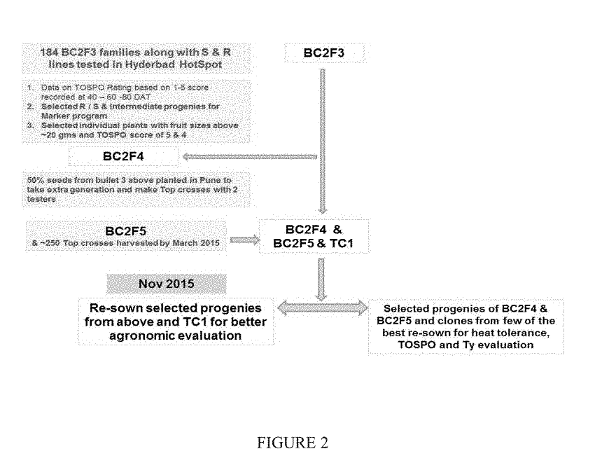

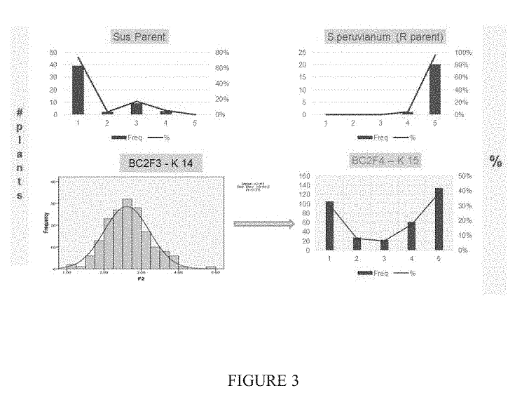

The S. peruvianum accession RP (used as donor), (S. peruvianum accession from AVRDC—Resistant to GBNV), was crossed with a GBNV susceptible S. lycopersicum line (SP1—that responds well to tissue culture). To overcome sexual incompatibility between S. peruvianum & S. lycopersicum, standard embryo rescue method was employed to raise the seven day old fertilized ovules on (FIG. 1 detailing the crossing and backcrossing) tissue culture medium. More than 5000 ovules were subjected to embryo rescue method and only two embryos were able to develop into true hybrid plants (F1) (seen phenotypically). These F1s were crossed with another GBNV susceptible line of S. lycopersicum as it was agronomically better & had more firm fruits (SP2—Susceptible parent 2 (Square Round)) and obtained BC1 seeds. The crossed ovules from these BC1 pollinations, using the inter-specific F1 as pollen source, were subjected to embryo rescue to get BC1 plants that were grown to fruiting. BC1F1 progeny was grown in the field in rainy season under high thrips pressure. Pollen from plants, that showed no GBNV symptoms were used to pollinate the SP1 recipient parent to obtain BC2F1 seeds. BC2F1 plants were grown and selfed to obtained BC2F2 seeds. Selected BC2F2 plants were grown to obtain BC2F3 (FIG. 2). Selected BC2F3 families were planted in hot spot location along with the parents (donor as well as recipient) in an augmented design where a susceptible check variety was planted repeatedly after every 10 rows in each bed. Frequency distribution of the disease in various generations and resistant (RP) and susceptible (SP) parent shown in FIG. 3.

### Example 2

Development of Field Screening for Disease and Disease Rating Scores:

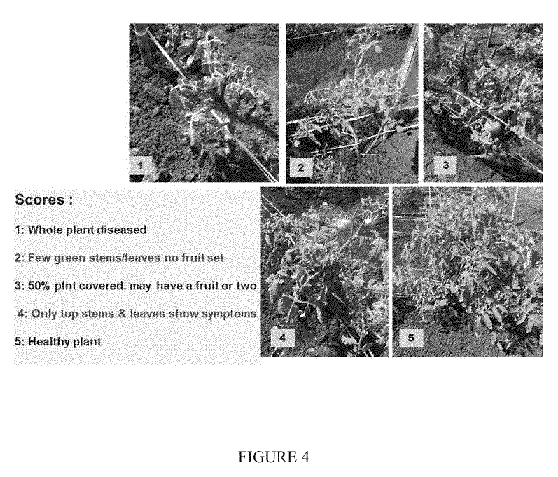

Using the phenotypic symptoms observed in the field at young and adult plant stages, e.g 40, 60, 80 days after transplantation in the field, the disease symptoms were categorized into 5 types as in FIG. 4 as (1) whole plant diseased (score 1); (2) diseased with few green stem and leaves (score 2); (3) 50% plant covered-may have one or two fruit (score 3); (4) Except for the top leaves, stem and other parts healthy (score 4); (5) Healthy plants (score 5).

Based on data on GBNV scores at 40, 60 & 80 days after transplanting (DAT) the families were grouped into three categories of: 1) Resistant (scores 4, 5); 2) intermediates (score 3) and 3) susceptible (scores 1 and 2). Three individual plants per category were harvested for obtaining BC2F4 seeds. BC2F4 families were again raised and BC2F5 progenies harvested from same groups of families.

The frequency distribution for disease score 1 to 5 for GBNV incidence on resistant parent, susceptible recipient, BC2F3 & BC2F4 progenies are provided in FIG. 3. Disease pressure during the screening has been very high as can be seen by the over 65 percent of susceptible parent shows susceptibility rating of 1 and 2 and another 15 percent having score of 3. The resistant parent on the other hand showed 90 percent plants scoring rating 5 and another 10 percent scoring a rating of 4, confirming high resistance in this parent (S. peruvianum). BC2F3 distribution for the disease rating shows a normal distribution (FIG. 3: bottom left) indicative of an incomplete dominance for resistance and involvement of more than one gene for its inheritance. BC2 F4 progenies derived from the susceptible and resistant families of BC2F3 showed the population moving towards resistant & susceptible groups (FIG. 3: bottom right). This indicates that it was possible to select high resistant genotypes with a disease rating scale of 4 & 5.

The mean disease ratings and their standard deviation in case of the BC2F3, BC2F4 & BC2F5 families versus resistant & susceptible parents as well as intermediates are presented in FIG. 3 below. These data represents three seasons of screening. BC2F5 progenies show an overall stable resistance which is closer to the donor parent while as the susceptible (recipient parent) and a super susceptible line as well as the progenies of intermediate resistance show clear susceptibility over all three seasons of testing. The BC2F5 progenies have further planted and the top stable lines for resistance to GBNV has been selected.

### Example 3

Genotyping, Linkage and QTL Analysis—Identification of Trait Linked Molecular Markers:

To understand precisely the location of resistance genes, 181 leaf samples consisting of 174 BC2F3 families (bulks of 4-20 plants) and 3 parental lines were used. DNA was extracted from each sample using standard extraction method.

For Marker identification SNP Panel was used
Solcap 7K Infinium SNP panel
6,007 SNPs resulted in genotypes with <10% missing datapoints
1,767 showed segregation in the population
556 SNPs selected for linkage construction and QTL analysis based on: Missing datapoints <20%
SNPs were observed to be polymorphic between SP2 and RP1 (original parents)

Trait linked molecular markers by way of multiple QTLs in specified chromosomes is identified. 7000 SNPs were generated and used to screen and identify the SNPs that were closely linked with the resistant trait. Out of 7000 SNPs that were used for the screening on the segregating materials, 17 SNPs were found to be associated with the resistance to GBNV trait.

The following were used for Linkage construction
556 SNPs used for linkage construction
330 SNPs were non-redundant (not co-segregated)
Mapped to 12 linkage groups
Linkage groups ranged from 79.2 cM to 174.9 cM
Total length 1,567.2 cM
Based on physical coordinates, linkage groups covered most of the genome

Out of 17 SNPs, 5 SNPs were found to closely linked to the resistance trait associated with two major QTLs present in chromosome 1 & 2.

### Example 4

Production of TOPSO Virus Resistant Tomato Line by Non-Transgenic Method: The BC2F6 progenies, resulting from individual plant selections (pedigree selection) since when the BC2F3 families were first screened in the rainy season in Hyderabad have shown high resistance levels to TOSPO during Kharif 16 at the hot spot locations. A summary data on the TOSPO scores of these progenies in K 16 vs checks and historically in their parent families are shown in table 1 below.

TABLE 1
TOSPO Resistant lines at BC2F6 vs susceptible (SP 1& 2) parents, Resistant (RP) parent
and a general susceptible check along with historical TOSPO scores over seasons

| | TOSPO scores over seasons | | | | | | |
|---|---|---|---|---|---|---|---|
| | | TOSPO Scr | K 16 | TOSPO Scr | | TOSPO Scr | |
| Kharif(K)16 | Source # | K 16 | Fruit Wt | K 16 | TOSPO Scr | K 15 | TOSPO Scr |
| code | in K15 | MEAN ± SD | (g) | (Lost due Flood) | K15 | Fruit Wt (g) | K14 |
| GB0001 | H2-7-2-1 | 4.69 ± 1.11 | 30 | 4.21 ± 0.97 | 3 ± 1.87 | 15 | 3.71 ± 1.43 |
| GB0024 | H17-5-1 | 4.83 ± 0.41 | 25 | 3.67 ± 1.12 | 1.6 ± 1.01 | 31 | 2.50 ± 1.46 |
| GB0027 | GB0049-1-SB | 4.75 ± 0.45 | 35 | 3.75 ± 0.71 | 5 ± 0 | 20 | 4.10 ± 1.16 |
| GB0038 | GB0720-1-2 | 4.36 ± 0.67 | 120 | 4 ± 0.76 | 5 ± 0 | | |
| GB0039 | GB0279-2-1 | 4.31 ± 0.48 | 50 | 4.15 ± 0.69 | 4.25 ± 1.50 | 50 | |
| SP1 | SP1 | 1.5 ± 0.22 | 60 | 1.6 ± 0.2 | 1.2 ± 0.18 | 55 | 1.5 ± 0.15 |
| SP2 | SP2 | 1.6 ± 0.18 | 65 | 1.3 ± 0.5 | 1.4 ± 0.28 | 60 | 1.6 ± 0.2 |
| RP | RP | 5 ± 0 | 5 | 4.9 ± 0.22 | 4.9 ± 0.24 | 5 | 4.8 ± 0.15 |
| GBE002 | GB0134-1 | 3.77 ± 1.17 | 80 | 4.27 ± 0.65 | 4 ± 0 | 15 | 4 |
| GBE003 | GB0134-3 | 4.73 ± 0.47 | 60 | 4 ± 0.77 | 4 ± 0 | 15 | 4 |
| GBE004 | GB0135-1 | 4 ± 1.11 | 20 | 4.80 ± 0.45 | 4.5 ± 0.57 | 20 | 4 |
| GBE009 | GB0202-4 | 3.91 ± 0.70 | 35 | 3.6 ± 1.12 | 5 ± 0 | 23 | 4 |
| GBE034 | GB0074-1 | 4.69 ± 0.48 | 100 | 4.07 ± 1.14 | 1.57 ± 1.51 | | 5 |
| GBE035 | GB0074-2 | 4.29 ± 0.47 | 90 | 4.27 ± 1.19 | 1.57 ± 1.51 | | 5 |
| GBE036 | GB0075-1 | 3.50 ± 1.07 | 35 | 5 ± 0 | 4 ± 2 | 15 | 5 |
| GBE040 | GB0196-1 | 4.46 ± 0.52 | 150 | 4 ± 1.58 | 1 ± 0 | | 4 |
| GBE062 | GB0703-2 | 3.88 ± 1.81 | 120 | 3.79 ± 1.72 | 5 ± 0 | | 5 |
| GBE064 | GB0704-1 | 4.64 ± 0.50 | 120 | 3.92 ± 1.31 | | | 5 |
| GBE073 | GB0708-1 | 4.86 ± 0.36 | 100 | 4.21 ± 0.97 | 4 ± 0 | | 5 |
| GBE074 | GB0709-1 | 4.62 ± 0.51 | 150 | 4.43 ± 1.09 | | | 5 |
| GBE075 | GB0710-1 | 4.64 ± 0.50 | 120 | 3.50 ± 0.84 | 5 ± 0 | | 5 |
| GBE083 | GB0782-1 | 4.55 ± 0.69 | 90 | 3.67 ± 0.50 | 5 ± 0 | | 5 |
| GBE084 | GB0782-BK | 4.83 ± 0.39 | 30 | 4.46 ± 0.66 | 5 ± 0 | | 5 |
| GBE085 | GB0783-1 | 4.85 ± 0.38 | 90 | 3.93 ± 1.21 | | | 5 |
| GBE086 | GB0783-BK | 4.90 ± 0.32 | 30 | 4.33 ± 0.98 | | | 5 |
| GBE087 | GB0784-BK | 4.08 ± 1.44 | 25 | 4.80 ± 0.42 | | | 5 |
| GBE088 | GB0785-BK | 4.43 ± 0.51 | 25 | 4 ± 0.67 | | | 5 |
| SEL-4 (S check) | SEL-4 | 1.71 ± 0.49 | 50 | 1.8 ± 0.56 | 2 ± 0.47 | | 1.7 ± 0.56 |

In FIG. 8 a few TOSPO resistant lines in field vs susceptible checks are presented

### Example 5

Production of TOPSO Virus Resistant Tomato Line by Marker Assisted Breeding

Following table show results of SNP marker analysis showing linkage with the TOSPO resistance trait.

TABLE 2
SNP association with resistance at 40 or 60 days

| | | Mean resistance Index | | | F-test | | | T-test | | |
|---|---|---|---|---|---|---|---|---|---|---|
| | SNP | A | B | H | A/B | A/H | B/H | A/B | A/H | B/H |
| QTL1 | SNP527 | 4.01 (209) | 4.44 (389) | 3.85 (117) | 0.0237 | 0.6354 | 0.0164 | 1E−06 | 0.1729 | 3E−07 |
| | SNP001 | 4.01 (215) | 4.43 (425) | 3.83 (116) | 0.0153 | 0.6749 | 0.0145 | 7E−07 | 0.1248 | 1E−07 |
| | SNP540 | 3.91 (267) | 4.52 (385) | 3.92 (103) | 2E−07 | 0.6044 | 0.001 | 8E−14 | 0.9208 | 4E−07 |
| | SNP066 | 3.98 (239) | 4.47 (405) | 3.82 (110) | 0.0064 | 0.2713 | 0.0008 | 2E−09 | 0.1754 | 7E−08 |
| | SNP521 | 3.82 (242) | 4.51 (430) | 3.79 (73) | 1E−05 | 0.2667 | 3E−05 | 3E−17 | 0.8699 | 2E−06 |
| | SNP434 | 3.89 (260) | 4.53 (390) | 3.7 (71) | 2E−08 | 0.2066 | 2E−07 | 8E−17 | 0.3148 | 4E−07 |
| QTL 2 | SNP044 | 3.52 (223) | 4.56 (484) | 4.04 (47) | 2E−15 | 0.0534 | 6E−14 | 3E−30 | 0.0034 | 0.0019 |
| | SNP017 | 3.5 (222) | 4.56 (481) | 4.13 (48) | 3E−15 | 0.4657 | 0.0004 | 5E−31 | 0.0004 | 0.0057 |
| | SNP304 | 3.5 (222) | 4.56 (481) | 4.13 (48) | 3E−15 | 0.4657 | 0.0004 | 5E−31 | 0.0004 | 0.0057 |
| | SNP131 | 3.51 (225) | 4.56 (479) | 4.18 (51) | 3E−15 | 0.3954 | 0.0004 | 2E−30 | 0.0001 | 0.0111 |
| | SNP155 | 3.71 (264) | 4.53 (430) | 4.26 (61) | 1E−15 | 0.1956 | 0.001 | 2E−22 | 0.0006 | 0.0474 |
| | SNP151 | 3.71 (263) | 4.53 (434) | 4.25 (57) | 2E−15 | 0.2413 | 0.0011 | 3E−22 | 0.0013 | 0.0432 |
| | SaNP193 | 3.69 (254) | 4.53 (437) | 4.2 (64) | 3E−16 | 0.1546 | 0.0007 | 1E−22 | 0.0012 | 0.0128 |
| | SNP097 | 3.69 (254) | 4.53 (437) | 4.2 (64) | 3E−16 | 0.1546 | 0.0007 | 1E−22 | 0.0012 | 0.0128 |
| | SNP034 | 3.5 (222) | 4.56 (485) | 4.13 (48) | 2E−15 | 0.4657 | 0.0003 | 4E−31 | 0.0004 | 0.0056 |
| Random | SNP506 | 3.62 (23) | 2.96 (8) | 3.48 (14) | 0.2265 | 0.2554 | 0.8112 | 0.0448 | 0.5999 | 0.2201 |
| SNP | SNP507 | 3.32 (16) | 3.53 (12) | 3.6 (16) | 0.4254 | 0.9966 | 0.4276 | 0.5264 | 0.3218 | 0.8441 |
| | SNP508 | 3.38 (16) | 3.41 (18) | 3.59 (11) | 0.9498 | 0.3004 | 0.2685 | 0.9134 | 0.4894 | 0.5577 |

SNP flanking the QTL1 and QTL2 were significantly associated with resistance index
Random SNP were not associated with the resistance index

Table 3 provides the impact of either or both QTLs

| | Resistance | F-test | | | T-test | | |
|---|---|---|---|---|---|---|---|
| QTL | index | QTL1 | QTL2 | QTL1/QTL2 | QTL1 | QTL2 | QTL1/QTL2 |
| — | 3.59 (86) | 0.124189557 | 0.356191085 | 1.11696E−07 | 0.029388201 | 0.001159854 | 6.99241E−14 |
| QTL1 | 3.24 (129) | | 0.007012633 | 5.22526E−18 | | 1.55039E−08 | 2.58405E−24 |
| QTL2 | 4.06 (112) | | | 9.24693E−06 | | | 5.14217E−08 |
| QTL1/QTL2 | 4.61 (438) | | | | | | |

Lines carrying QTL2 had significantly higher resistance index than the lines carrying QTL1
Lines carrying both QTL had significantly higher resistance index than either QTL

1. A method for producing a tomato plant, wherein the tomato plant exhibits TOSPO Virus resistance, comprising:
(a) providing a donor tomato plant, wherein the donor plant exhibits TOSPO virus resistance;
(b) transferring at least one nucleic acid from the first tomato plant to one or more tomato plants, wherein the recipient tomato plants do not exhibit TOSPO virus resistance, and wherein the transfer results in the introduction of genomic material from the donor tomato plant in a corresponding region of one or more recipient tomato plants; and
(c) identifying and selecting, from the one or more recipient tomato plants, a tomato plant that comprises within its genome at least one quantitative trait locus (QTL) for TOSPO virus resistance, wherein the QTL is selected from the group consisting of: QTL1, QTL2 and TOSPO viral resistance related traits thereof, and wherein QTL1 is indicated by a genomic region on chromosome 1 and QTL2 is indicated by a genomic region on chromosome 2.

2. The method for producing the tomato plant of claim 1, further comprising the step of selecting a recipient tomato plant which is infected by TOSPO Viruses

3. The method of claim 1, wherein the recipient tomato plant exhibits one or more of the traits selected from deformed shape and uneven ripening/yellow patch formation on the fruit, wilting of the top portion of the plant, dark brown spots on the leaves and dark brown patches on stems of the plants.

4. The method of claim 1, wherein the transfer of one or more nucleic acids is performed by crossing the donor tomato plant with a recipient tomato plant to produce progeny tomato plants comprising at least one QTL for TOSPO virus resistance as in introgression, and wherein step (c) is performed on one or more progeny plants.

5. The method of claim 1, wherein step (c) is performed by detecting the marker in DNA isolated from the recipient tomato plants.

6. The method of claim 1, wherein step (c) is performed by detecting the marker in DNA isolated from one or more progeny plants.

7. The method of claim 1, wherein step (c) comprises selecting a recipient tomato plant that comprises within its genome at least two QTLs selected from the group consisting of QTL1, QTL 2 and virus-resistance related parts thereof.

8. The method of claim 1, wherein step (c) comprises selecting a recipient tomato plant that comprises within its genome at least two QTLs selected from the group consisting of QTL1, QTL2 and virus-resistance related parts thereof.

9. The method of claim 1, wherein step (c) comprises selecting a recipient tomato plant that comprises QTL1 and QTL2 within its genome.

10. The method of claim 1, wherein step (c) comprises selecting a recipient tomato plant that comprises within its genome QTL or a viral resistance related part thereof, and at least one QTL selected from the group consisting of QTL1, QTL2 and related parts thereof.

11. The method of claim 1, wherein the method further comprises a step of crossing a recipient tomato plant selected in step (c) with a plant of a cultivated variety of tomato to produce progeny plants.

12. The method of claim 11, wherein the recipient tomato plant selected in step (c) is of a same variety as the plant of the cultivated variety of tomato to which the recipient tomato plant is crossed.

13. The method of claim 1, wherein the TOSPO virus is a Groundnut bud necrosis virus (GBNV).

14. A tomato plant exhibiting TOSPO (GBNV) virus resistance, produced by a method of claim 1, wherein the tomato plant or part thereof comprises a QTL selected from the group consisting of QTL1, QTL2 and TOSPO (GBNV) virus related parts thereof, wherein the QTLs or viral resistance-related parts thereof are not present in the natural genetic background of the tomato plants.

15. A tomato plant exhibiting TOSPO (GBNV) virus resistance, produced by crossing the tomato plant selected in step (c) of claim 1 with a tomato plant that exhibits commercially desirable characteristics, wherein the resulting hybrid plant comprises at least one QTL selected from the group consisting of QTL1, QTL2 and TOSPO virus resistance related parts thereof, wherein at least one QTL is not present in the natural background of either the tomato plant selected in step (c) or the tomato plant exhibiting commercially desirable characteristics.

16. The tomato plant of claim 15, wherein at least one resistance-related QTL is from Solanum peruvianum.

17-19. (canceled)

20. A seed of the plant of claim 15.

21. The tomato plant of claim 15, wherein the TOSPO virus is a Groundnut bud necrosis virus (GBNV).
